SEQUENCE LISTING (8 sequences)

SEQ ID NO: 1 - DNA, 1464 bp, Human herpesvirus 4
  CDS (1)..(1464)
atgcctacattctatcttgcgttacatgggggacaaacatatcatctaattgttgacacggatagtcttggaaacccgtcactctcagtaattccctcgaatccctaccaggaacaactgtcagacactccattaattccactaacaatctttgttggggaaaacacgggggtgcccccaccactcccaccaccccccccaccaccacccccaccacccccaccacccccaccacccccaccacccccaccacctccaccaccttcaccaccacccccgcccccaccacccccaccacctcagcgcagggatgcctggacacaagagccatcacctcttgatagggatccgctaggatatgacgtcgggcatggacctctagcatctgctatgcgaatgctttggatggctaattatattgtaagacaatcacggggtgaccggggccttattttgccacaaggcccacaaacagcccctcaggccgtgttggtacagccacatgtcccccctctacgcccgacagcacccaccattttgtcacctctgtcacaaccgaggcttacccctccacaaccactcatgatgccatcaaggcctacccctcctacccctctgccacctgcaacactaacggtgccaccaaggcctacccgtcctaccactctgccacccacaccactactcacggtactacaaaggcctaccgaacttcaacccacaccatcaccaccacgcatgcatctccctgtcttgcatgtgccagaccaatcaatgcaccctcttactcatcaaagcaccccaaatgatccagatagtccagaaccacggtccccgactgtattttataacattccacctatgccattacccccctcacaattgccaccaccagcagcaccagcacagccacctccaggggtcatcaacgaccaacaattacatcatctaccctcggggccaccatggtggccacccatctgcgaccccccgcaaccctctaagactcaaggccagagccggggacagagcagggggaggggcaggggcaggggcaggggcaggggcaagggcaagtccagggacaagcaacgcaagcccggtggaccttggagaccagagccaaacacctccagtcctagcatgcctgaactaagtccagtcctcggtcttcatcagggacaaggggctggggactcaccaactcctggcccatccaatgccgcccccgtttgtagaaattcacacacggcaacccctaacgtttcaccaatacatgaaccggagtcccataatagcccagaggctcccattctcttccccgatgattggtatcctccatctatagaccccgcagacttagacgaaagttgggattacatttttgagacaacagaatctcctagctcagatgaagattatgtggagggacccagtaaaagacctcgcccctccatccagtaa

SEQ ID NO: 2 - protein, 487 aa, Human herpesvirus 4
MPTFYLALHGGQTYHLIVDTDSLGNPSLSVIPSNPYQEQLSDTPLIPLTIFVGENTGVPPPLPPPPPPPPPPPPPPPPPPPPPPPPPPSPPPPPPPPPPPQRRDAWTQEPSPLDRDPLGYDVGHGPLASAMRMLWMANYIVRQSRGDRGLILPQGPQTAPQAVLVQPHVPPLRPTAPTILSPLSQPRLTPPQPLMMPSRPTPPTPLPPATLTVPPRPTRPTTLPPTPLLTVLQRPTELQPTPSPPRMHLPVLHVPDQSMHPLTHQSTPNDPDSPEPRSPTVFYNIPPMPLPPSQLPPPAAPAQPPPGVINDQQLHHLPSGPPWWPPICDPPQPSKTQGQSRGQSRGRGRGRGRGRGKGKSRDKQRKPGGPWRPEPNTSSPSMPELSPVLGLHQGQGAGDSPTPGPSNAAPVCRNSHTATPNVSPIHEPESHNSPEAPILFPDDWYPPSIDPADLDESWDYIFETTESPSSDEDYVEGPSKRPRPSIQ

SEQ ID NO: 3 - DNA, 1464 bp, Human herpesvirus 4
atgcctacattctatcttgcgttacatgggggacaaacatatcatctaattgttgacacggatagtcttggaaacccgtcactctcagtaattccctcgaatccctaccaggaacaactgtcagacactccattaattccactaacaatctttgttggggaaaacacgggggtgcccccaccactcccaccaccccccccaccaccacccccaccacccccaccacccccaccacccccaccacccccaccacctccaccaccttcaccaccacccccgcccccaccacccccaccacctcagcgcagggatgcctggacacaagagccatcacctcttgatagggatccgctaggatatgacgtcgggcatggacctctagcatctgctatgcgaatgctttggatggctaattatattgtaagacaatcacggggtgaccggggccttattttgccacaaggcccacaaacagcccctcaggccaggttggtccagccacatgtcccccctctacgcccgacagcacccaccattttgtcacctctgtcacaaccgaggcttacccctccacaaccactcatgatgccaccaaggcctacccctcctacccctctgccacctgcaacactaacggtgccaccaaggcctacccgtcctaccactctgccacccacaccactactcacggtactacaaaggcctaccgaacttcaacccacaccatcaccaccacgcatgcatctccctgtcttgcatgtgccagaccaatcaatgcaccctcttactcatcaaagcaccccaaatgatccagatagtccagaaccacggtccccgactgtattttataacattccacctatgccattacccccctcacaattgccaccaccagcagcaccagcacagccacctccaggggtcatcaacgaccaacaattacatcatctaccctcggggccaccatggtggccacccatctgcgaccccccgcaaccctctaagactcaaggccagagccggggacagagcagggggaggggcaggggcaggggcaggggcaggggcaagggcaagtccagggacaagcaacgcaagcccggtggaccttggagaccagagccaaacacctccagtcctagcatgcctgaactaagtccagtcctcggtcttcatcagggacaaggggctggggactcaccaactcctggcccatccaatgccgcccccgtttgtagaaattcacacacggcaacccctaacgtttcaccaatacatgaaccggagtcccataatagcccagaggctcccattctcttccccgatgattggtatcctccatctatagaccccgcagacttagacgaaagttgggattacatttttgagacaacagaatctcctagctcagatgaagattatgtggagggacccagtaaaagacctcgcccctccatccagtaa

SEQ ID NO: 4 - DNA, 1467 bp, Human herpesvirus 4
  CDS (1)..(1467)
atgcctacattctatcttgcgttacatgggggacaaacatatcatctaattgttgacacggatagtcttggaaacccgtcactctcagtaattccctcgaatccctaccaggaacaactgtcagacactccattaattccactaacaatctttgttggggaaaacacgggggtgcccccaccactcccaccaccccccccaccaccacccccaccacccccaccacccccaccacccccaccacccccaccacctccaccaccttcaccaccacccccgcccccaccacccccaccacctcagcgcagggatgcctggacacaagagccatcacctcttgatagggatccgctaggatatgacgtcgggcatggacctctagcatctgctatgcgaatgctttggatggctaattatattgtaagacaatcacggggtgaccggggccttattttgccacaaggcccacaaacagcccctcaggccgtgttggtacagccacatgtcccccctctacgcccgacagcacccaccattttgtcacctctgtcacgaccgaggcttacccctccacaaccactcatgattccaccaaggcctacccctccttcccctctgccacctgcaacactactcacggtgccaccaaggcctacccgtcctaccactttgccacccacaccactactcacggtactacaaaggcctaccgaacttcaacccacaccatcaccaccacgcatgcatctccctgtcttgcatgtgccagaccaatcaatgcaccctcttactcatcaaagcaccccaaatgatccagatagtccagaaccacggtccccgactgtattttataacattccacctatgccattacccccctcacaattgccaccaccagcagcaccagcacagccacctccaggggtcatcaacgaccaacaattacatcatctaccctcggggccaccatggtggccacccatctgcgaccccccgcaaccctctaagactcaaggccagagccggggacagagcagggggaggggcaggggcaggggcaggggcaggggcaagggcaagtccagggacaagcaacgcaagcccggtggaccttggagaccagagccaaacacctccagtcctagcatgcctgaactaagtccagtcctcggtcttcatcagggacaaggggctggggactcaccaactcctggcccatccaatgccgcccccgtttgtagaaattcacacacggcaacccctaacgtttcaccaatacatgaaccggagtcccataatagcccagaggctcccattctcttccccgatgattggtatcctccatctatagaccccgcagacttagacgaaagttgggattacatttttgagacaacagaatctcctagctcagatgaagattatgtggagggacccagtaaaagacctcgcccctccatccagtaa

SEQ ID NO: 5 - protein, 488 aa, Human herpesvirus 4
MPTFYLALHGGQTYHLIVDTDSLGNPSLSVIPSNPYQEQLSDTPLIPLTIFVGENTGVPPPLPPPPPPPPPPPPPPPPPPPPPPPPPPSPPPPPPPPPPPQRRDAWTQEPSPLDRDPLGYDVGHGPLASAMRMLWMANYIVRQSRGDRGLILPQGPQTAPQAVLVQPHVPPLRPTAPTILSPLSRPRLTPPQPLMIPPRPTPPSPLPPATLLTVPPRPTRPTTLPPTPLLTVLQRPTELQPTPSPPRMHLPVLHVPDQSMHPLTHQSTPNDPDSPEPRSPTVFYNIPPMPLPPSQLPPPAAPAQPPPGVINDQQLHHLPSGPPWWPPICDPPQPSKTQGQSRGQSRGRGRGRGRGRGKGKSRDKQRKPGGPWRPEPNTSSPSMPELSPVLGLHQGQGAGDSPTPGPSNAAPVCRNSHTATPNVSPIHEPESHNSPEAPILFPDDWYPPSIDPADLDESWDYIFETTESPSSDEDYVEGPSKRPRPSIQ

SEQ ID NO: 6 - DNA, 20 bp, Artificial Sequence
  PRIMER E2C
agggatgcctggacacaaga

SEQ ID NO: 7 - DNA, 20 bp, Artificial Sequence
  PRIMER E2SEQ4
gtaatggcataggtggaatg

SEQ ID NO: 8 - DNA, 20 bp, Artificial Sequence
  PRIMER 2A.2
ttctggactatctggatcat

(19) United States
(12) Patent Application Publication
(10) Pub. No.: US 2016/0060718 A1
(43) Pub. Date: Mar. 3, 2016

(54) EPSTEIN BARR VIRUS GENOTIPIC VARIANTS AND USES THEREOF AS RISK PREDICTORS, BIOMARKERS AND THERAPEUTIC TARGETS IN MULTIPLE SCLEROSIS

(71) Applicant: UNIVERSITÀ DEGLI STUDI DI ROMA "LA SAPIENZA", Roma (RM) (IT)

(72) Inventors: Giovanni RISTORI, Rome (IT); Rosella MECHELLI, Rome (IT); Marco SALVETTI, Rome (IT); Claudia POLICANO, Rome (IT); Renato UMETON, Rome (IT)

(21) Appl. No.: 14/782,556

(22) PCT Filed: Apr. 7, 2014
(86) PCT No.: PCT/IB2014/060489

§ 371 Date: Oct. 5, 2015

(30) Foreign Application Priority Data
Apr. 5, 2013 (IT) RM2013A000206

(51) Int. Cl. C12Q 1/70; C07K 14/005

(52) U.S. Cl. CPC C12Q 1/705; C07K 14/005; C12Q 2600/172; C12Q 2600/156; C12Q 2600/118; C12N 2710/16222

(57) ABSTRACT

The present invention relates to a nucleic acid coding for a variant of the Epstein Barr nuclear antigen 2 (EBNA2) for use as a biomarker for predicting the risk of developing multiple sclerosis and/or for screening and/or for the diagnosis and/or prognosis of multiple sclerosis, and to an in vitro method for predicting the risk of developing and/or for screening for multiple sclerosis and/or for the diagnosis and/or prognosis of multiple sclerosis in a subject, comprising the detection of the presence of said nucleic acid.

### FIELD OF THE INVENTION

The present invention lies in the medical field, and in particular relates to an in vitro method for detecting the presence of genotype variants of the Epstein Barr virus (EBV), and to the use thereof as possible risk predictors, biomarkers, and therapeutic target in multiple sclerosis.

### PRIOR ART

Multiple sclerosis (MS) is a demyelinating disease of the central nervous system, it belongs to the group of autoimmune diseases and has unknown aetiology, however there is evidence of an association both with genetic and environmental factors.

Epidemiological, clinical and laboratory studies support the aetiological role of the Epstein Barr virus (EBV) in MS¹. In fact, human beings are the only natural host of the virus, which may explain why MS is a pathology which manifests itself exclusively in humans.

The studies carried out until now have shown that the onset of infectious mononucleoses and primary infection at young-adult age correlate with an increased risk of developing multiple sclerosis: in Northern Europe (in areas with a high prevalence of MS), a third of the population is infected with EBV during or after puberty, whilst in Southern Europe (areas in which the prevalence is low), subclinical infections occur in the first years of life. In addition, seroepidemiological evidence suggests an aetiological role for the virus: perspective studies have demonstrated that the increase of serum antibody titres against the virus precede the onset of multiple sclerosis. Viral proteins are a target of the antibody response detected in the cerebrospinal fluid of patients, and post-mortem studies have revealed the infection by EBV in the cerebral tissues of the affected subjects.

Patent application WO2008/125366 describes that the infection of the B cells by EBV leads to autoimmune diseases, including multiple sclerosis, and proposes the use of anti-EBV substances for the treatment of the autoimmune diseases.

However, the discrepancy between global spread of the infection (more than 90% of the adult population) and the relatively limited prevalence of MS remains a large obstacle for interpreting the pathogenetic mechanisms forming the basis of the association between the ubiquitous virus and the disease. The presence of genotype variants of the virus specifically associated with the pathology can contribute to the explanation of this paradox.

Given the methodological difficulties in the study of the variants of EBV through sequencing of the whole viral genome, an approach focused on a particular gene (candidate-gene approach) is followed by various groups²⁻⁷.

In the gene coding for the latent protein of the “Epstein Barr nuclear antigen 2” (EBNA2) resides one of the most variable regions of the entire virus, which allows to discriminate between the two main types of EBV (type 1 and 2). In addition, EBNA2 is expressed during the first phase of infection of the B cells, having a key role in the activation and in the proliferation of these cells.

Five major variants of the type 1 virus (the most widespread in the Caucasian population)⁸ have been identified within the sequence of EBNA2⁹ defining the following alleles: 1.1, 1.2, 1.3A, 1.3B and 1.3E. These variants are characterised by changes with respect to the sequence of the reference prototype B95.8 (defined as allele 1.1) in the most polymorphic region (B95.8 coordinates 48.959-49.208) of the coding portion of EBNA2.

Previous studies²⁻⁷, based on multiple sclerosis, have analysed other regions of the virus without revealing particular correlations between multiple sclerosis and the gene variants of EBV.

There is thus a need to provide a method for identifying subjects who are “at risk” or pathological situations having diagnostic or prognostic peculiarities, and for identifying therapeutic targets, also for preventative strategies.

### DESCRIPTION OF THE INVENTION

The authors have identified a connection between MS and gene variants of EBV. The authors have investigated the link between the variants of EBNA2 and MS in a population of continental Italy. The present invention is based on the identification of gene variants of the Epstein Barr virus (EBV), identified in the coding sequence of the Epstein Barr nuclear antigen 2 (EBNA2) protein, in a population of subjects affected by multiple sclerosis (MS). The peculiarity of the EBV virus genotype that characterises the subjects with MS compared to controls paired by age, sex and geographical area can be utilised as a potential biomarker (risk-predictive condition for the development of the disease, a trait that may contribute to the diagnosis and/or prognosis of MS).

The authors have demonstrated a correlation between an EBV-associated pathology, in particular MS, and viral gene variants in the region of EBNA2, selected for being notoriously the most polymorphic region of the viral genome.

The “profile” of the gene variants of EBNA2 may contribute to the identification of subjects who are “at risk” or pathological situations having specific diagnostic or prognostic peculiarities.

In addition, the gene variants identified by the authors can be used as a therapeutic target, even for preventative therapies. For example, such variants can be used to produce anti-viral drugs or vaccines.

Object of the present invention is a compound consisting of:

a) a nucleic acid coding for the variant of the 1.2 sub-type of Epstein Barr nuclear antigen 2 (EBNA2), characterised in that it comprises at least one substitution of the triplet coding for one or more amino acids selected from the group consisting of:

aa. 134, aa. 236, aa. 245 and aa. 267 of SEQ ID NO: 2, or

b) a messenger RNA transcribed from said nucleic acid, or

c) a protein coded by said nucleic acid.

The nucleotide sequence of EBNA2-1.2 (SEQ ID NO: 1) is as follows:

| | atgcctacat tctatcttgc gttacatggg ggacaaacat atcatctaat tgttgacacg |
|---|---|
| 61 | gatagtcttg gaaacccgtc actctcagta attccctcga atccctacca ggaacaactg |
| 121 | tcagacactc cattaattcc actaacaatc tttgttgggg aaaacacggg ggtgccccca |
| 181 | ccactcccac cacccccccc accaccaccc ccaccacccc caccaccccc accaccccca |
| 241 | ccacccccac cacctccacc accttcacca ccacccccgc ccccaccacc cccaccacct |
| 301 | cagcgcaggg atgcctggac acaagagcca tcacctcttg atagggatcc gctaggatat |
| 361 | gacgtcgggc atggacctct agcatctgct atgcgaatgc tttggatggc taattatatt |
| 421 | gtaagacaat cacggggtga ccggggcctt attttgccac aaggcccaca aacagcccct |
| 481 | caggccgtgc tggtacagcc acatgtcccc cctctacgcc cgacagcacc caccattttg |
| 541 | tcacctctgt cacaaccgag gcttacccct ccacaaccac tcatgatgcc atcaaggcct |
| 601 | acccctccta cccctctgcc acctgcaaca ctaacggtgc caccaaggcc tacccgtcct |
| 661 | accactctgc cacccacacc actactcacg gtactacaaa ggcctaccga acttcaaccc |
| 721 | acaccatcac caccacgcat gcatctccct gtcttgcatg tgccagacca atcaatgcac |
| 781 | cctcttactc atcaaagcac cccaaatgat ccagatagtc cagaaccacg gtccccgact |
| 841 | gtattttata acattccacc tatgccatta cccccctcac aattgccacc accagcagca |
| 901 | ccagcacagc cacctccagg ggtcatcaac gaccaacaat tacatcatct accctcgggg |
| 961 | ccaccatggt ggccacccat ctgcgacccc ccgcaaccct ctaagactca aggccagagc |
| 1021 | cggggacaga gcagggggag gggcaggggc aggggcaggg gcaggggcaa gggcaagtcc |
| 1081 | agggacaagc aacgcaagcc cggtggacct tggagaccag agccaaacac ctccagtcct |
| 1141 | agcatgcctg aactaagtcc agtcctcggt cttcatcagg gacaaggggc tggggactca |
| 1201 | ccaactcctg gcccatccaa tgccgccccc gtttgtagaa attcacacac ggcaacccct |
| 1261 | aacgtttcac caatacatga accggagtcc cataatagcc cagaggctcc cattctcttc |
| 1321 | cccgatgatt ggtatcctcc atctatagac cccgcagact tagacgaaag ttgggattac |
| 1381 | atttttgaga caacagaatc tcctagctca gatgaagatt atgtggaggg acccagtaaa |
| 1441 | agacctcgcc cctccatcca gtaa |

The amino acid sequence of EBNA2-1.2 (SEQ ID NO: 2) is as follows:

```
                10 20 30 40 50 60
MYTFYLALHG GQTYHLIVDT DSLGNPSLSV IPSNPYQEQL SDTPLIPLTT FVGENTGVPP
                70 80 90 100 110 120
PLPPPPPPPP PPPPPPPPPP PPPPPPPPSP PPPPPPPPPP QRRDAWTQEP SPLDRDPLGY
              130 140 150 160 170 180
DVGHGPLASA MRMLWMANYI VRQSRGDRGL ILPQGPQTAP QAVLVQPHVP PLRPTAPTIL
              190 200 210 220 230 240
SPLSQPRLTP PQPLMMPSRP TPPTPLPPAT LTVPPRPTRP TTLPPTPLLT VLQRPTELQP
              250 260 270 280 290 300
TPSPPRMHLP VLHVPDQSMH PLTHQSTPND PDSPEPRSPT VFYNIPPMPL PPSQLPPPAA
              310 320 330 340 350 360
PAQPPPGVIN DQQLHHLPSG PPWWPPICDP PQPSKTQGQS RGQSRGRGRG RGRGRGKGKS
              370 380 390 400 410 420
RDKQRKPGGP WRPEPNTSSP SMPELSPVLG LHQGQGAGDS PTPFPSNAAP VCRNSHTATP
              430 440 450 460 470 480
NVSPIHEPES HNSPEAPILF PDDWYPPSID PADLDESWDY IFETTESPSS DEDYVEGPSK
RPRPSIQ
```

In a preferred embodiment the protein is coded by a nucleic acid wherein said substitution of the triplet coding for at least one of the amino acids selected from the group consisting of: aa. 134, aa. 236, aa. 245 and aa. 267 of SEQ ID NO: 2 does not correspond to a silent mutation. More preferably, said protein in position aa. 245 presents an amino acid S or T and/or in position aa. 267 presents an amino acid I.

In a preferred aspect of the invention the substitution of the triplet corresponds to at least one of the following:

the substitution of the triplet CTT with the triplet CTG coding for aa. 134 of SEQ ID NO: 2, the substitution of the triplet ACC with the triplet ACT coding for aa. 236 of SEQ ID NO: 2, the substitution of the triplet CCA with the triplet TCA or ACA coding for aa. 245 of SEQ ID NO: 2, the substitution of the triplet ACC with the triplet ATC coding for aa. 267 of SEQ ID NO: 2.

More preferably, said substitution of the triplet corresponds to at least the substitution of the triplet ACC with the triplet ATC coding for aa. 267 of SEQ ID NO: 2 and/or the substitution of the triplet CTT with the triplet CTG coding for aa. 134 of SEQ ID NO: 2. The compound according to the invention is preferably a biomarker of multiple sclerosis.

A further object of the invention is a compound consisting of:

a) a nucleic acid coding for a variant of the Epstein Barr nuclear antigen 2 (EBNA2), characterised in it comprises:

the sequence coding for a nuclear antigen 2 of the sub-type 1.2 or variants thereof, characterised by at least one substitution of the triplet coding for one or more amino acids selected from the group consisting of: aa. 134, aa. 236, aa. 245 and aa. 267 of SEQ ID NO: 2, or

b) a messenger RNA transcribed from said nucleic acid, or

c) a protein coded by said nucleic acid

for use in a method for predicting the risk of contracting or developing multiple sclerosis and/or for screening and/or for the diagnosis and/or the prognosis of multiple sclerosis. Said compound is preferably a biomarker of multiple sclerosis.

Said substitution of the triplet preferably corresponds to at least one of the following:

the substitution of the triplet CTT with the triplet CTG coding for aa. 134 of SEQ ID NO: 2, the substitution of the triplet ACC with the triplet ACT coding for aa. 236 of SEQ ID NO: 2, the substitution of the triplet CCA with the triplet TCA or ACA coding for aa. 245 of SEQ ID NO: 2, the substitution of the triplet ACC with the triplet ATC coding for aa. 267 of SEQ ID NO: 2.

More preferably, the substitution of the sequence corresponds to at least the substitution of the triplet ACC with the triplet ATC coding for aa. 267 of SEQ ID NO: 2 and/or the substitution of the triplet CTT with the triplet CTG coding for aa. 134 of SEQ ID NO: 2.

The compound of the present invention, DNA, RNA or protein, finds industrial applicability, for example in the identification of primers and/or probes or as an antigenic compound.

A further object of the invention is an in vitro method for predicting the risk of contracting or developing multiple sclerosis in a subject, wherein the detection of the compound as above defined, in a biological sample isolated from the subject, is an indication of increased risk.

In a preferred embodiment of the method according to the present invention, the presence of the nucleic acid as defined above is detected through DNA genotyping. In a preferred aspect of the method according to the invention, the detection of the presence of the sequence coding for the nuclear antigen 2 of the sub-type 1.3 B or of the protein coded by it is an indication of low risk.

The nucleotide sequence of EBNA2-1.3B (SEQ ID NO: 4) is as follows:

atgcctacat tctatcttgc gttacatggg ggacaaacat atcatctaat tgttgacacg
gatagtcttg gaaacccgtc actctcagta attccctcga atccctacca ggaacaactg
tcagacactc cattaattcc actaacaatc tttgttgggg aaaacacggg ggtgccccca
ccactcccac cacccccccc accaccaccc ccaccacccc caccaccccc accaccccca
ccacccccac cacctccacc accttcacca ccacccccgc ccccaccacc cccaccacct
cagcgcaggg atgcctggac acaagagcca tcacctcttg atagggatcc gctaggatat
gacgtcgggc atggacctct agcatctgct atgcgaatgc tttggatggc taattatatt
gtaagacaat cacggggtga ccggggcctt attttgccac aaggcccaca aacagcccct
caggccgtgt tggtacagcc acatgtcccc cctctacgcc cgacagcacc caccattttg
tcacctctgt cacgaccgag gcttacccct ccacaaccac tcatgattcc accaaggcct
acccctcctt cccctctgcc acctgcaaca ctactcacgg tgccaccaag gcctacccgt
cctaccactt tgccacccac accactactc acggtactac aaaggcctac cgaacttcaa
cccacaccat caccaccacg catgcatctc cctgtcttgc atgtgccaga ccaatcaatg
caccctctta ctcatcaaag caccccaaat gatccagata gtccagaacc acggtccccg
actgtatttt ataacattcc acctatgcca ttacccccct cacaattgcc accaccagca
gcaccagcac agccacctcc aggggtcatc aacgaccaac aattacatca tctaccctcg
gggccaccat ggtggccacc catctgcgac cccccgcaac cctctaagac tcaaggccag
agccggggac agagcagggg gaggggcagg ggcaggggca ggggcagggg caagggcaag
tccagggaca agcaacgcaa gcccggtgga ccttggagac cagagccaaa cacctccagt
cctagcatgc ctgaactaag tccagtcctc ggtcttcatc agggacaagg ggctggggac
tcaccaactc ctggcccatc caatgccgcc cccgtttgta gaaattcaca cacggcaacc
cctaacgttt caccaataca tgaaccggag tcccataata gcccagaggc tcccattctc
ttccccgatg attggtatcc tccatctata gaccccgcag acttagacga aagttgggat
tacatttttg agacaacaga atctcctagc tcagatgaag attatgtgga gggacccagt
aaaagacctc gcccctccat ccagtaa

The amino acid sequence of EBNA2-1.3B (SEQ ID NO: 5) is as follows:

```
MPTFYLALHG GQTYHLIVDT DSLGNPSLSV IPSNPYQEQL SDTPLIPLTI FVGENTGVPP
                70 80 90 100 110 120
PLPPPPPPPP PPPPPPPPPP PPPPPPPPSP PPPPPPPPPP QRRDAWTQEP SPLDRDPLGY
              130 140 150 160 170 180
DVGHGPLASA MRMLWMANYI VRQSRGDRGL ILPQGPQTAP QAVLVQPHVP PLRPTAPTIL
              190 200 210 220 230 240
SPLSQPRLTP PQPLMIPPRP TPPSPLPPAT LLTVPPRPTRP TYLPPTPLLT VLQRPTELQP
              250 260 270 280 290 300
TPSPPRMHLP VLHVPDQSMH PLTHQSTPND PDSPEPRSPT VFYNIPPMPL PPSQLPPPAA
              310 320 330 340 350 360
PAQPPPGVIN DQQLHHLPSG PPWWPPICDP PQPSKTQGQS RGQSRGRGRG RGRGRGKGKS
              370 380 390 400 410 420
RDKQRKPGGP WRPEPNTSSP SMPELSPVLG LHQGQGAGDS PTPGPSNAAP VCRNSHTATP
              430 440 450 460 470 480
NVSPIHEPES HNSPEAPILF PDDWYPPSID PADLDESWDY IFETTESPSS DEDYVEGPSK
RPRPSIQ
```

Another object of the invention is an in vitro method for screening and/or for the diagnosis and/or prognosis of multiple sclerosis in a subject through detection of the presence of the compound as defined above, in a biological sample isolated from the subject. In a preferred embodiment of the method according to the present invention, the presence of the nucleic acid as defined above is detected through DNA genotyping.

In the present invention a “method for screening” or the “screening” preferably includes the screening of subjects potentially at risk for multiple sclerosis.

In a preferred embodiment of the in vitro method according to the invention, the subject in which the presence of the compound as defined above has been detected is then subjected to further methods for diagnosis and/or prognosis of multiple sclerosis, including, for example, magnetic resonance of the brain and of the spinal cord, and examination of the cerebrospinal fluid. Such diagnostic methods currently used are costly and invasive. Thus, the method according to the invention allows to carry out a first screening of subjects potentially at risk that is effective, fast and non-invasive.

In the methods according to the present invention, the biological sample is preferably selected from: blood cells, biological fluids, preferably serum, plasma, saliva or cerebrospinal fluid, cerebral tissue, epithelial cells. The biological sample can also be any cell type able to be infected by the Epstein Barr virus.

A further object of the invention is a kit for predicting the risk of contracting or developing multiple sclerosis and/or for screening and/or diagnosis and/or prognosis of multiple sclerosis comprising the detection of the compound as defined above, comprising detection means for said compound and optionally control means.

In the kit according to the invention, the detected compound is preferably a nucleic acid, and the kit further comprises means for amplifying said nucleic acid.

The control means can be used to compare the presence of the compound as defined above with a proper control. The control can be obtained for example, with reference to the known standards, either from a normal subject or from a normal population. If the compound is a protein, the detection means are, for example, at least one antibody specific for the protein, functional analogues or derivatives thereof.

In the present invention, the detection of the compound as defined above may refer to the detection of the presence of one, two, three, four, five, six, etc. compounds as defined above. Any combination of the compounds is suitable for the purpose of the invention.

A further object of the invention is an inhibitor for the Epstein Barr virus having as a specific target the compound as defined above, for use in the prevention and/or treatment of multiple sclerosis, said inhibitor preferably being a vaccine, an antibody, a siRNA or a drug with low atomic weight.

The variants described above may comprise one of the substitutions of the triplets mentioned above, or two of the substitutions of the triplets mentioned above (for example the substitution of the triplets coding for aa. 134 and 236, aa. 134 and 245, aa. 134 and 267, aa. 236 and 245, aa. 236 and 267, aa. 245 and 267), or three of the substitutions of the triplets mentioned above (for example the substitution of the triplets coding for aa. 134, 236 and 245; for aa. 134, 236 and 267; for aa. 134, 245 and 267; for aa. 236, 245 and 267), or four of the substitutions of the triplets mentioned above (for example the substitution of the triplets coding for aa. 134, 236, 245 and 267).

The variants of the nucleotide sequence EBNA2-1.2 are preferably characterised in that they have a nucleotide substitution from T to G in position 402 of SEQ ID No. 1 and/or a nucleotide substitution of a C with a T in position 708 of SEQ ID No. 1 and/or a nucleotide substitution of a C with an A or a T in position 733 of SEQ ID No. 1, and/or a nucleotide substitution of a C with a T in position 800 of SEQ ID No. 1.

The variant of the amino acid sequence EBNA2-1.2 corresponding to the nucleotide sequence where the nucleotide substitution of a C with a T or with an A is present in nucleotide position 733 of SEQ ID No. 1 is characterised in that it has in position aa 245 of SEQ ID No. 2, respectively, an amino acid S or T, instead of an amino acid P. The variant of the amino acid sequence EBNA2-1.2 corresponding to the nucleotide sequence wherein in the nucleotide position 800 of SEQ ID No. 1, is present a nucleotide substitution of a C with a T is characterised in that it has in position aa 267 of SEQ ID No. 2 an amino acid I instead of an amino acid T.

In the present invention, the expression “detection” in relation to a protein or to a nucleic acid (DNA or RNA) refers, for example, to any method of observation, ascertainment, or quantification of the signals indicative of the presence of a protein in a sample or the absolute or relative quantity of said target protein in a sample. The methods may be combined with methods for labelling proteins or nucleic acids for providing a signal, for example: immunohistochemical staining, ELISA, cell suspension, cytology, fluorescence, radioactivity, colorimetry, gravimetry, X-ray diffraction or adsorption, magnetism, enzyme activity and the like. In the present invention, the detection of the presence of messenger RNA transcribed from the nucleic acid as defined above can be performed by means of any technique known to a person skilled in the art, for example Northern blotting or quantitative or semi-quantitative RT-PCR methods using suitable oligonucleotide primers. DNA detection can be carried out for example through DNA genotyping. For DNA genotyping is intended any technique which allows to detect the genotype from an organism by means of biological tests (genotype tests). These techniques include, for example: PCR, analysis of a DNA fragment, allele-specific oligonucleotide (ASO) probes, DNA sequencing and microarray.

The present invention will be described in non-limiting examples with reference to the following figure:

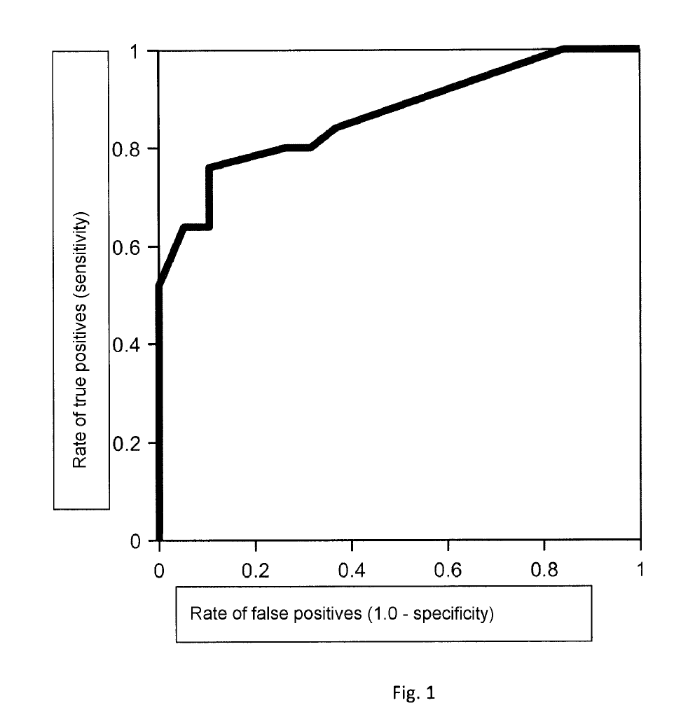

FIG. 1: ROC (receiver operating characteristic) curve generated using a Bayes classification approach which takes into consideration the sex of the subject and the genotype of EBNA2 (Epstein Barr virus nuclear antigen 2). The area under the curve (AUC) is equal to 0.87; the AUC precision recall=0.84; F measure=0.77.

### EXAMPLES

Subjects and Methods

Blood samples were taken from patients with relapsing-remitting multiple sclerosis MS¹¹ and healthy donors (HD) matched by age, sex and geographical origin. At the same time as the blood sampling, the patients underwent magnetic resonance imaging (MRI) with contrast agent (gadolinium). The Ethics Committee approved the study, and all the participating subjects gave written informed consent.

Peripheral blood mononuclear cells (PBMC) were obtained by density centrifugation over Ficoll-Hypaque according to standard procedure. The cells were stained with human anti-CD19 antibodies (Miltenyi Biotec) and separated by magnetic beads in accordance with the manufacturer's recommended protocol.

Genomic DNA was extracted from PBMCs, CD19+ B cells and cell lines (B95-8 EBV-positive cell line and BJAB EBV-negative cell line) by a commercially available kit (QIAmp DNA mini kit, Qiagen). All the samples were analysed by a “semi-nested PCR” approach using specific primers of EBNA2⁹:

PRIMER E2C:
(SEQ ID NO: 6)
AGGGATGCCTGGACACAAGA
PRIMER E2SEQ4:
(SEQ ID NO: 7)
GTAATGGCATAGGTGGAATG
PRIMER 2A.2:
(SEQ ID NO: 8)
TTCTGGACTATCTGGATCAT

All PCR products were assayed by means of sequence analyses.

The sequences were aligned with the multiple sequence alignment program ClustalW2 (www.ebi.ac.uk/Tools/msa/clustalw2/) and the variants were considered when there was a deviation from EBV B95.8 prototype (NCBI accession number V01555; V01555-EBNA-2 (SEQ ID NO: 3, published):

| | |
|---|---|
| 48503 | atgccta cattctatct tgcgttacat gggggacaaa |
| 48541 | catatcatct aattgttgac acggatagtc ttggaaaccc gtcactctca gtaattccct |
| 48601 | cgaatcccta ccaggaacaa ctgtcagaca ctccattaat tccactaaca atctttgttg |
| 48661 | gggaaaacac gggggtgccc ccaccactcc caccaccccc cccaccacca cccccaccac |
| 48721 | ccccaccacc cccaccaccc ccaccacccc caccacctcc accaccttca ccaccacccc |
| 48781 | cgcccccacg acccccacca cctcagcgca gggctgcctg gacacaagag ccatcacctc |
| 48841 | ttgataggga tccgctagga tatgacgtcg ggcatggacc tctagcatct gctatgcgaa |
| 48901 | tgctttggat ggctaattat attgtaagac aatcacgggg tgaccggggc cttattttgc |
| 48961 | cacaaggccc acaaacagcc cctcaggcca ggttggtcca gccacatgtc ccccctctac |
| 49021 | gcccgacagc acccaccatt ttgtcacctc tgtcacaacc gaggcttacc cctccacaac |
| 49081 | cactcatgat gccaccaagg cctacccctc ctacccctct gccacctgca acactaacgg |
| 49141 | tgccaccaag gcctacccgt cctaccactc tgccacccac accactactc acggtactac |
| 49201 | aaaggcccac cgaacttcaa cccacaccat caccaccacg tatgcatctc cctgtcttgc |
| 49261 | atgtgccaga ccaatcaatg caccctctta ctcatcaaag caccccaaat gatccagata |
| 49322 | gtccagaacc acggtccccg actgtatttt ataacattcc acctatgcca ttacccccct |
| 49381 | cacaattgcc accaccagca gcaccagcac agccacctcc aggggtcatc aacgaccaac |
| 49441 | aattacatca tctaccctcg gggccaccat ggtggccacc catctgcgac cccccgcaac |
| 49501 | cctctaagac tcaaggccag agccggggac agagcagggg gaggggcagg ggcaggggca |
| 49561 | ggggcagggg caagggcaag tccagggaca agcaacgcaa gcccggtgga ccttggagac |
| 49621 | cagagccaaa cacctccagt cctagcatgc ctgaactaag tccagtcctc ggtcttcatd |
| 49681 | agggacaagg ggctggggac tcaccaactc ctggcccatc caatgccgcc cccgtttgta |
| 49741 | gaaattcaca cacggcaacc cctaacgttt caccaataca cgaaccggag tcccataata |
| 49801 | gcccagaggc tcccattctc ttccccgatg attggtatcc tccatctata gaccccgcag |
| 49861 | acttagacga aagttgggat tacatttttg agacaacaga atctcctagc tcagatgaag |
| 49921 | attatgtgga gggacccagt aaaagacctc gcccctccat ccagtaa |

All the participants of the study (patients and healthy subjects) were subjected to human leukocyte antigen (HLA) loci typing. The haplotypes of classes I HLA-A, HLA-B and HLA-C and of class II DRB1 were analysed by means of standard sequence specific primer polymerase chain reaction (SSP-PCR)¹², using “Histo Type DNA” wells plates (BAG, Formedic diagnostici, Milan, Italy) in accordance with manufacturer's instructions.

It was possible to detect the alleles recognised by the specific primers after amplification in a GeneAmp PCR 9700 thermocycler (Applied Biosystems, Foster City, Calif. U.S.A.) and gel electrophoresis on 2% agarose. Fisher's exact test (Graph Pad Prism 5) was used to compare the proportion of variants in all patients vs HD, whilst Barnard's exact test¹³ was used to compare only the donors infected with the sub-type 1.2.

A Bayes classifier¹⁴ was used for partitioning the dataset: 50% of the data was used to train the classifier, and the remaining 50% was then used to test its predictive capability. The receiver operating characteristic (ROC) curve and the area under the curve (AUC) were then computed to quantify the predictive potential of EBNA2 genotyping as a marker of disease status.

Results

By means of “semi-nested PCR”, the authors amplified a fragment of approximately 500 bp corresponding to the fragment having coordinates 48.810-49.310 located within the hypervariable region of EBNA2, which was then subjected to sequencing.

Blood samples were obtained from 58 patients affected by MS (6 undergoing immunomodulatory therapy) and 49 healthy donors (HD) selected by age, sex, ethnicity and geographic origin, comparable with the group of patients. The success rate of the EBNA2 genotyping was comparable between the two groups: 53/91 (58%) in the group of patients and 37/56 in the healthy subjects (66%).

Of the 53 patients with a successful genotyping, 7 were under therapy with Interferon beta, whilst the others had never been treated. The mean age was 35±10.7 in the patients and 37±9.5 in the HD; the male/female ratio was 13/40 in the patients, 11/26 in the controls. Seven patients with MS and three HD were analysed at different time points (from one to twelve months between each sampling): two patients and one HD presented infection from various strains (1.2, 1.3B, GD1) (Table 3) at different time points, whilst the majority of the donors showed stability of the EBV sub-type.

Considering each sampling as an independent observation, a total of 107 blood samples (66 blood samples from patients with MS and 41 from HD) were analysed.

A significant difference in the distribution of the EBV sub-types was observed between patients and controls (Table 1): the allele 1.2 was prevalent in the patients with MS (26/53 vs 6/37, p=0.0004), whereas the allele 1.3B was prevalent in the controls (26/37 vs 18/53, p=0.0012; Fisher's exact test). When the samples obtained from the same subject at different time points were added to the analyses (66 from patients with MS and 41 from HD), comparable results in the EBV sub-types and in the distribution of the variants were obtained, as well as in the potential of predicting the state of the disease (shown in Tables 1-2).

The obtained data was analysed by comparing the distribution of the viral strains between healthy or diseased women and men, observing a distribution comparable with that obtained over the entire population. The gender therefore did not appear to modify the aforementioned distribution (Table 1B).

Extending the observation beyond the region 48990-49170 (where the variants described in literature reside)⁹, new sequence variations, never observed before, were identified, in correspondence of the amino acids 134, 236, 245, 256 and 267 (Table 2; Table 3). Whilst the variation at position 256 (Asp/Asn) is present in sub-types 1.2, 1.3B and GD1, without differences between patients and controls, the other four variants resulted detectable only in the presence of the sub-type 1.2, and therefore correlated with MS (Table 2; Table 3).

To verify the consistency of the EBNA2 genotyping data, a Bayes classifier¹⁴ was tested for its capability to predict the state of the disease. The predictive potential of the EBV genetic variants was therefore quantified and illustrated in a ROC curve (FIG. 1): the AUC was 0.87, a value which is more than good in accordance with broadly accepted ROC efficiency metrics¹⁴.

In general, the risk of developing the disease is higher in carriers of sub-type 1.2 (odd ratio[OR]=5) and variants thereof (in particular the divergence of amino acid 245, OR=9.4), whilst the presence of sub-type 1.3B demonstrated to be protective, OR=0.22.

No correlation was found between EBV genetic variants and HLA haplotypes (included those known to be associated with MS)¹⁵ of the donors. No correlation was found between EBV genotypes and the clinical characteristics of the patients (MRI data, disability scales, disease duration).

The subjects included in this study were characterised by the HLA haplotype and by their clinical and neuroradiological characteristics at the moment of blood sampling. The authors have demonstrated that the state of the disease correlates with an excess of EBNA2 sub-type 1.2 and a deficiency of sub-type 1.3B, irrespective of the clinical characteristics of the patients and their HLA haplotype. In addition, the patients have a greater probability of being infected with viral strains presenting new genotype variants correlated to sub-type 1.2 (in particular that corresponding at amino acid position 245).

Previous studies on a possible link between EBV genotypes and MS have provided conflicting results: no link with EBNA6,² EBNA1 and LMP1,^{3,4} EBNA2;⁵ “marginally different frequencies” for BRRF2 and EBNA1;⁶ most frequent co-infection EBNA2 of type 1 and 2 in patients compared with the healthy subjects.⁷

Differences in the geographic variants can explain, in part, the incoherencies, as has been reported for malignant EBV-correlated tumours and the different geographical distribution thereof.¹⁶

In a region of EBNA2 not studied by the previous studies, the authors found genotypes with associations that are stronger than those reported beforehand: for a model that includes EBNA2 sub-types and new identified EBNA2 polymorphisms, the ROC analysis provides increased reliability in the prediction of the state of the MS.

Aside from the predictive potential of the EBNA2 variants, the probability that they can induce functional consequences, contributing in this way to the aetiology of the disease, merits further consideration. In a virus with a low propensity to mutate, new variants have greater probability of having a certain functional impact insofar as they tend to be maintained and fixed in the viral genome.

There are only two main types of EBV (type 1 and 2), which seem to be identical for the majority of the genome, but demonstrate allele polymorphism in a sub-group of latent genes: EBNA-LP, EBNA2, EBNA3A, EBNA3B and EBNA3C. In particular, the two types of EBV share 64% of the nucleotide sequences and 53% of the amino acids sequences predicted of EBNA2.¹⁷ EBNA2 sequence mutations are able to influence its interaction with other host proteins.¹⁸

The authors studied the most polymorphic region of EBNA2, where the sequence divergence between type 1 and type 2 resides. These differences can have immunological consequences.^{19,20}

In addition, this region is involved in interactions with cellular proteins, such as Nur77²¹ and SMARCB1²², that have been associated respectively with MS²³ and antiviral responses.²⁴

These results are complementary to data showing that part of the genetic predisposition to MS can be attributable to variants in genes that interact with EBV.

TABLE 1
Frequency of the assessed EBNA2 sub-types in
peripheral blood of MS and HD divided by subjects and
samples (in 10 subjects we have multiple determinations of
the genotype obtained at different time points).

| | | MS (%) | HD SUBJECTS | | MS (%) | SAMPLES HD | |
|---|---|---|---|---|---|---|---|
| Type | Sub-type | N = 53 | (%) N = 37 | p-value* | N = 66 | (%) N = 41 | p-value* |
| B95-8 | 1.1 | 0 | 0 | / | 0 | 0 | / |
| B95-8 | 1.2 | 26 (49) | 6 (16) | 0.0004 | 35 (53) | 9 (22) | 0.0022 |
| B95-8 | 1.3B | 18 (34) | 26 (70) | 0.0012 | 21 (32) | 27 (66) | 0.0007 |
| B95-8 | 1.3A | 5 (9) | 0 | n.s. | 5 (7.5) | 0 | n.s. |
| GD1 | / | 4 (8) | 5 (14) | n.s. | 5 (7.5) | 5 (12) | n.s. |

EBNA2 = Epstein Barr nuclear antigen 2;
aa = amino acid;
MS = multiple sclerosis;
HD = healthy donors;
n.s. = not significant.

TABLE 2
Frequency of the EBNA2 variants of new
identification in peripheral blood of assessed MS and HD
divided by subjects and samples(in 10 subjects we have
multiple determinations of the genotype obtained at
different time points).

| | | | | | | | SAMPLES | |
|---|---|---|---|---|---|---|---|---|
| aa | | | MS (%) | HD SUBJECTS | | MS (%) | HD (%) | p- |
| position | B95-8 | Variant | (N = 53) | (%) N = 37 | p-value* | N = 66 | N = 41 | value* |
| 134 | L(CTT) | L(CTG) | 25 (47) | 6 (16) | 0.0032 | 33 (55) | 8 (19.5) | 0.002 |
| 236 | T(ACC) | T(ACT) | 23 (43) | 6 (16) | 0.011 | 32 (48.5) | 9 (22) | 0.0077 |
| | | S(TCA) | 11 (21) | 1 (3) | 0.0132 | 17 (26) | 1 (2.4) | 0.0012 |
| 245 | P(CCA) | | | | | | | |
| | | T(ACA) | 6 (11) | 3 (8) | n.s. | 6 (9) | 4 (10) | n.s. |
| 256 | D(GAC) | N(ACC) | 10 (19) | 9 (24) | n.s. | 12 (18) | 10 (24) | n.s. |
| 267 | T(ACC) | I(ATC) | 25 (47) | 6 (16) | 0.0032 | 34 (51) | 8 (19.5) | 0.0011 |

EBNA2 = Epstein bar nuclear antigen 2;
aa = amino acid;
MS = multiple sclerosis;
HD = healthy donors;
n.s. = not significant.

TABLE 3
Nucleotide and amino acid substitutions within the
EBNA2 sequence compared with the genomes of type 1 B95-8
and GD1.

| | F/M | aa134 | aa163 | aa165 | aa185 | aa196 | aa198 | aa204 | INS | a223 | aa236 | aa245 | aa256 | aa267 | Strain |
|---|---|---|---|---|---|---|---|---|---|---|---|---|---|---|---|
| | — | L(CTT) | R | V(GTC) | Q | M | P | T | — | L(CTG) | T(ACC) | P | D | T | B95 |
| | | | | | | | | | | | | | | | -8 |
| | — | L(CTT) | M | V(GTA) | Q | M | P | T | — | L(CTG) | T(ACC) | P | D | T | GD1 |
| HD1 | F | | V | V(GTA) | R | I | | S | L | L(TTG) | | | | | 1.3B |
| HD2 | F | | V | V(GTA) | R | I | | S | L | L(TTG) | | | | | 1.3B |
| HD2-1 | M | | V | V(GTA) | R | I | | S | L | L(TTG) | | | | | 1.3B |
| HD2-3 | | | V | V(GTA) | R | I | | S | L | L(TTG) | | | | | 1.3B |
| HD4 | F | | M | V(GTA) | | | | | | | | | | | GD1 |
| HD5-1 | M | L(CTG) | V | V(GTA) | | | S | | | | T(ACT) | | N | I | 1.2 |
| HD5-2 | | L(CTG) | V | V(GTA) | | | S | | | | T(ACT) | | N | I | 1.2 |
| HD5-3 | | no seq | V | V(GTA) | | | S | | | | T(ACT) | | | I | 1.2 |
| HD6 | M | | M | V(GTA) | | | | | | | | | | | GD1 |
| HD7-1 | M | L(CTG) | V | V(GTA) | | | S | | | | T(ACT) | T | | no seq | 1.2 |
| HD7-2 | | | V | V(GTA) | R | I | | S | L | L(TTG) | | | | | 1.3B |
| HD8 | F | | | | R | I | | S | L | L(TTG) | | | | | 1.3B |
| HD9 | F | | V | V(GTA) | R | I | S | S | L | L(TTG) | | | | | 1.3B |
| HD10 | F | | V | V(GTA) | R | I | | S | L | L(TTG) | | | | | 1.3B |
| HD11 | F | | V | V(GTA) | R | I | | S | L | L(TTG) | | | | | 1.3B |
| HD12 | M | | V | V(GTA) | R | I | | S | L | L(TTG) | | | | | 1.3B |
| HD13 | F | L(CTG) | V | V(GTA) | | | S | | | | T(ACT) | T | N | I | 1.2 |
| HD14 | M | | V | V(GTA) | R | I | | S | L | L(TTG) | | | N | | 1.3B |
| HD15 | F | | V | V(GTA) | R | I | | S | L | L(TTG) | | | N | | 1.3B |
| HD16 | F | | M | V(GTA) | | | | | | | | | N | | GD1 |
| HD17 | M | L(CTG) | V | V(GTA) | | | S | | | | no seq | no seq | no seq | no seq | 1.2 |
| HD18 | F | | V | V(GTA) | R | I | | S | L | L(TTG) | | | | | 1.3B |
| HD19 | M | L(CTG) | V | V(GTA) | | | S | | | | T(ACT) | S | | I | 1.2 |
| HD20 | F | | V | V(GTA) | R | I | | S | L | L(TTG) | | | | | 1.3B |
| HD21 | F | L(CTG) | V | V(GTA) | | | S | | | | T(ACT) | | | I | 1.2 |
| HD22 | F | | V | V(GTA) | R | I | | S | L | L(TTG) | | | | | 1.3B |
| HD23 | F | | V | V(GTA) | R | I | | S | L | L(TTG) | | | N | | 1.3B |
| HD24 | F | | V | V(GTA) | R | I | | S | L | L(TTG) | | | | | 1.3B |
| HD25 | M | | V | V(GTA) | R | I | | S | L | L(TTG) | | | N | | 1.3B |
| HD26 | F | | V | V(GTA) | R | I | | S | L | L(TTG) | | | | | 1.3B |
| HD27 | F | | V | V(GTA) | R | I | | S | L | L(TTG) | | | | | 1.3B |
| HD28 | F | | M | V(GTA) | | | | | | | | | | | GD1 |
| HD29 | F | | V | V(GTA) | R | I | | S | L | L(TTG) | | | | | 1.3B |
| HD30 | F | | V | V(GTA) | R | I | | S | L | L(TTG) | | | | | 1.3B |
| HD31 | F | | V | V(GTA) | R | I | | S | L | L(TTG) | | | S | | 1.3B |
| HD32 | F | | V | V(GTA) | R | I | | S | L | L(TTG) | | | | | 1.3B |
| HD33 | M | | M | V(GTA) | | | | | | | | | S | | GD1 |
| HD34 | F | | V | V(GTA) | R | I | | S | L | L(TTG) | | | | | 1.3B |
| HD35 | F | | V | V(GTA) | R | I | | S | L | L(TTG) | | | | | 1.3B |
| HD36 | F | L(CTG) | V | V(GTA) | | | S | | | | T(ACT) | T | | I | 1.2 |
| HD37 | M | | | V(GTA) | R | I | | S | L | L(TTG) | | | | | 1.3B |
| MS3-1 | F | L(CTG) | V | V(GTA) | | | S | | | | T(ACT) | S | | I | 1.2 |
| MS3-2 | | L(CTG) | V | V(GTA) | | | S | | | | T(ACT) | S | | I | 1.2 |
| MS3-3 | | no seq | V | V(GTA) | | | S | | | | T(ACT) | S | | I | 1.2 |
| MS3 | F | | V | V(GTA) | R | I | | S | L | L(TTG) | | T | | | 1.2 |
| MS3 | M | | M | V(GTA) | | | | | | | | | N | | 1.2 |
| MS4-1 | M | L(CTG) | V | V(GTA) | | | S | | | | T(ACT) | S | | I | 1.2 |
| MS4-2 | | L(CTG) | V | V(GTA) | | | S | | | | T(ACT) | S | | I | 1.2 |
| MS4-3 | | L(CTG) | V | V(GTA) | | | S | | | | T(ACT) | S | | I | 1.2 |
| MS5-1 | M | L(CTG) | V | V(GTA) | | | S | | | | T(ACT) | S | | I | 1.2 |
| MS5-2 | | L(CTG) | V | V(GTA) | | | S | | | | T(ACT) | S | N | I | 1.2 |
| MS5-3 | | L(CTG) | V | V(GTA) | | | S | | | | T(ACT) | S | | I | 1.2 |
| MS6-1 | F | | V | V(GTA) | R | I | | S | L | L(TTG) | | | | | 1.3B |
| MS6-2 | | | V | V(GTA) | R | I | | S | L | L(TTG) | | | | | 1.3B |
| MS6-3 | | L(CTG) | V | V(GTA) | | | S | | | | T(ACT) | S | | I | 1.2 |
| MS7-1 | F | L(CTG) | V | V(GTA) | | | S | | | | T(ACT) | | | I | 1.2 |
| MS7-3 | | L(CTG) | V | V(GTA) | | | S | | | | T(ACT) | | N | I | 1.2 |
| MS8-1 | M | L(CTG) | V | V(GTA) | | | S | | | | T(ACT) | | | I | 1.2 |
| MS8-3 | | L(CTG) | V | V(GTA) | | | S | | | | T(ACT) | | | I | 1.2 |
| MS8-3 | | L(CTG) | V | V(GTA) | | | S | | | | T(ACT) | | | I | 1.2 |
| MS9 | F | | V | V(GTA) | R | I | | S | L | L(TTG) | | | | | 1.3B |
| MS10-1 | F | | V | V(GTA) | R | I | | S | L | L(TTG) | | | | | 1.3B |
| MS10-2 | | | M | V(GTA) | | | | | | | | | | | GD1 |
| MS10-3 | | | V | V(GTA) | | | S | | | | | no seq | | no seq | 1.2 |
| MS11 | M | L(CTG) | V | V(GTA) | | | S | | | | T(ACT) | S | | I | 1.2 |
| MS12 | F | | V | V(GTA) | R | I | | S | L | L(TTG) | | | | | 1.3B |
| MS13 | M | L(CTG) | V | V(GTA) | | | S | | | | | T | | I | 1.2 |
| MS14 | F | | M | V(GTA) | | | | | | | | | N | | GD1 |
| MS15 | F | L(CTG) | V | V(GTA) | | | S | | | L(TTG) | | T | N | I | 1.2 |
| MS16 | F | | V | | R | I | | S | L | | | | | | 1.3B |
| MS17 | F | | V | V(GTA) | R | I | | S | L | L(TTG) | | | | | 1.3B |
| MS18 | F | L(CTG) | V | V(GTA) | | | S | | | | T(ACT) | S | | I | 1.2 |
| MS19 | F | L(CTG) | V | V(GTA) | | | S | | | | T(ACT) | S | | I | 1.2 |
| MS20 | F | L(CTG) | V | V(GTA) | | | S | | | | T(ACT) | S | | I | 1.2 |
| MS21 | F | | V | V(GTA) | R | I | | S | no seq | no seq | | | N | | 1.3B |
| MS22 | F | L(CTG) | V | V(GTA) | | | S | | | | T(ACT) | S | N | I | 1.2 |
| MS23 | F | | V | V(GTA) | R | I | | S | L | L(TTG) | | | N | | 1.3B |
| MS24 | F | L(CTG) | V | V(GTA) | | | S | | | | T(ACT) | T | | I | 1.2 |
| MS25 | F | L(CTG) | V | V(GTA) | | | S | | | | T(ACT) | | | I | 1.2 |
| MS26 | M | L(CTG) | V | V(GTA) | | | S | | | | T(ACT) | S | N | I | 1.2 |
| MS27 | F | | V | V(GTA) | R | I | | S | L | L(TTG) | | | | | 1.3A |
| MS28 | M | | V | V(GTA) | R | I | | S | L | L(TTG) | | | N | | 1.3B |
| MS29 | F | L(CTG) | V | V(GTA) | | | S | | | | T(ACT) | | | I | 1.2 |
| MS30 | F | L(CTG) | V | V(GTA) | | | S | | | | T(ACT) | S | | I | 1.2 |
| MS31 | F | | V | V(GTA) | R | I | | S | L | L(TTG) | | | | | 1.3B |
| MS32 | F | | V | V(GTA) | R | I | | S | L | L(TTG) | | | N | | 1.3B |
| MS33 | F | | V | V(GTA) | R | I | | S | L | L(TTG) | | | | | 1.3B |
| MS34 | F | L(CTG) | V | V(GTA) | | | S | | | | T(ACT) | T | N | I | 1.2 |
| MS35 | F | | V | V(GTA) | R | I | | S | L | L(TTG) | | | | | 1.3B |
| MS36 | F | | V | V(GTA) | R | I | | S | L | L(TTG) | | | | | 1.3A |
| MS37 | M | | M | V(GTA) | | | | | | | | | | | GD1 |
| MS38 | F | | M | V(GTA) | | | | | | | | | | | GD1 |
| MS39 | F | | V | V(GTA) | R | I | | S | L | L(TTG) | | | | | 1.3A |
| MS40 | F | | V | V(GTA) | R | I | | S | | L(TTG) | | | | | 1.3B |
| MS41 | F | | V | V(GTA) | R | I | | S | L | L(TTG) | | | | | 1.3B |
| MS42 | F | | V | V(GTA) | R | I | | S | L | L(TTG) | | | | | 1.3B |
| MS43 | M | | V | V(GTA) | R | I | | S | L | L(TTG) | | | | | 1.3B |
| MS44 | M | | V | V(GTA) | R | I | | S | | L(TTG) | | | | | 1.3B |
| MS45 | F | L(CTG) | V | V(GTA) | | | S | | | | T(ACT) | | | I | 1.2 |
| MS46 | F | L(CTG) | V | V(GTA) | | | S | | | | T(ACT) | | | I | 1.3 |
| MS47 | F | L(CTG) | V | V(GTA) | | | S | | | | T(ACT) | | | I | 1.3 |
| MS48 | F | L(CTG) | V | V(GTA) | | | S | | | | T(ACT) | | | I | 1.3 |
| MS49 | F | L(CTG) | V | V(GTA) | | | S | | | | T(ACT) | T | | I | 1.3 |
| MS50 | M | | V | V(GTA) | R | I | | S | L | L(TTG) | | | | | 1.3B |
| MS51 | M | | V | V(GTA) | R | I | | S | L | L(TTG) | | | | | 1.3B |
| MS52 | F | L(CTG) | V | V(GTA) | | | S | | | | T(ACT) | | | I | 1.3 |
| MS53 | F | | V | V(GTA) | R | I | | S | | L(TTG) | | | | | 1.3A |

INS = amino acid insertion;
HD = healthy donors;
MS = patients with multiple sclerosis.

### BIBLIOGRAPHY

Lünemann J D. Epstein-Barr virus in multiple sclerosis: a continuing conundrum. Neurology 2012; 78:11-12.
2. Munch M, Hvas J, Christensen T, Møller-Larsen A et al. A single subtype of Epstein Barr virus in members of multiple sclerosis clusters. Acta Neurol Scand. 1998; 98:395-9
3. Lindsey J W, Patel S, Zou J. Epstein-Barr virus genotypes in multiple sclerosis. Acta Neurol Scand. 2008; 117:141-4
4. Simon K C, Yang X, Munger K L, Ascherio A, EBNA1 and LMP1 variants in multiple sclerosis cases and controls. Acta Neurol Scand. 2011; 124:53-8
5. Lay M L, Lucas R M, Toi C, Ratnamohan M, et al. Epstein-Barr Virus Genotypes and Strains in Central Nervous System Demyelinating Disease and Epstein-Barr Virus-Related Illnesses in Australia. Intervirology. 2012 Jan. 24. [Epub ahead of print]
6. Brennan R M, Burrows J M, Bell M J, Bromham L, et al. Strains of Epstein-Barr virus infecting multiple sclerosis patients. Mult Scler. 2010; 16:643-51
7. Santòn A, Cristóbal E, Aparicio M, Royuela A et al. High frequency of co-infection by Epstein-Barr virus types 1 and 2 in patients with multiple sclerosis. Mult Scler. 2011; 17:1295-300
8. Yao Q Y, Croom-Carter D S, Tierney R J, Habeshaw G, et al. Epidemiology of infection with Epstein-Barr virus types 1 and 2: lessons from the study of a T-cell-immunocompromised hemophilic cohort. J Virol. 1998; 72:4352-63
9. Tierney R J, Edwards R H, Sitki-Green D, Croom-Carter D, et al. Multiple Epstein-Barr virus strains in patients with infectious mononucleosis: comparison of ex vivo samples with in vitro isolates by use of heteroduplex tracking assays. J Infect Dis. 2006; 193:287-97
10. Henkel T, Lind P D, Hayward S D & Peterson M G. Mediation of Epstein-Barr virus EBNA2 transactivation by recombination signal-binding protein J kappa. Science 1994; 265:92-95
11. Polman C H, Reingold S C, Banwell B, Clanet M, et al. Diagnostic criteria for multiple sclerosis. 2010 revisions to the McDonald criteria. Ann Neurol 2011; 69:292-302
12. Olerup O, Zetterquist H, HLA-DR typing by PCR amplification with sequence-specific primers (PCR-SSP) in 2 hours: an alternative to serological DR typing in clinical practice including donor-recipient matching in cadaveric transplantation. Tissue Antigens. 1992; 39:225-35
13. Barnard, G A. A New Test for 2×2 Tables. Nature 1945; 156:177.
14. Freidman N, Geiger D, Goldszmidt M. Bayesian Network Classifiers. Machine Learning 1997; 29:131.
15. International Multiple Sclerosis Genetics Consortium; Wellcome Trust Case Control Consortium 2, Sawcer S, et al. Genetic risk and a primary role for cell-mediated immune mechanisms in multiple sclerosis. Nature. 2011; 476:214
16. Sawada A, Croom-Carter D, Kondo O, Yasui M, et al Epstein-Barr virus latent gene sequences as geographical markers of viral origin: unique EBNA3 gene signatures identify Japanese viruses as distinct members of the Asian virus family. J Gen Virol. 2011; 92:1032-43
17. Sample J, Young L, Martin B, Chatman T, et al. Epstein-Barr virus types 1 and 2 differ in their EBNA-3A, EBNA-3B, and EBNA-3C genes. J Virol. 1990; 64:4084-92
18. Ling P D, Hayward S D. Contribution of conserved amino acids in mediating the interaction between EBNA2 and CBF1/RBPJk. J Virol. 1995; 69:1944-50
19. Klein G, Klein E, Kashuba E. Interaction of Epstein-Barr virus (EBV) with human B lymphocytes. Biochem Biophys Res Commun. 2010; 396:67-73
20. Lucchesi W, Brady G, Dittrich-Breiholz O, Kracht M, et al. Differential gene regulation by Epstein-Barr virus type 1 and type 2 EBNA2. J Virol. 2008; 82:7456-66
21. Lee J M, Lee K H, Farrell C J, Ling P D, et al. EBNA2 is required for protection of latently Epstein-Barr virus-infected B cells against specific apoptotic stimuli. J Virol. 2004; 78:12694-7
22. Wilson B G, Roberts C W. SWI/SNF nucleosome remodellers and cancer. Nat Rev Cancer. 2011; 11:481-92
23. Achiron A. Feldman A, Gurevich M. Characterization of the multiple sclerosis traits: nuclear receptors (NR) impaired apoptosis pathway and the role of 1-alpha 25-dihydroxyvitamin D(3). J Neurol Sci. 2011; 311:9-14
24. Cui K, Tailor P, Liu H, Chen X, et al. The chromatin-remodeling BAF complex mediates cellular antiviral activates by promoter priming. Mol Cell Biol. 2004; 24:4476-86

1. A compound consisting of:
a) a nucleic acid coding for the variant of the 1.2 sub-type of Epstein Barr nuclear antigen 2 (EBNA2), comprising a substitution of one nucleotide in the triplet coding for one or more amino acids selected from the group consisting of: aa. 134, aa. 236, aa. 245 and aa. 267 of SEQ ID NO: 2,
b) a messenger RNA transcribed from said nucleic acid, and
c) a protein coded by said nucleic acid.

2. The compound according to claim 1, wherein the substitution of the triplet corresponds to at least one of the following: the substitution of the triplet CTT with the triplet CTG coding for aa. 134 of SEQ ID NO: 2, the substitution of the triplet ACC with the triplet ACT coding for aa. 236 of SEQ ID NO: 2, the substitution of the triplet CCA with the triplet TCA or ACA coding for aa. 245 of SEQ ID NO: 2, the substitution of the triplet ACC with the triplet ATC coding for aa. 267 of SEQ ID NO: 2.

3. The compound according to claim 2, wherein the substitution of the triplet corresponds to at least the substitution of the triplet ACC with the triplet ATC coding for aa. 267 of SEQ ID NO: 2 and/or the substitution of the triplet CTT with the triplet CTG coding for aa. 134 of SEQ ID NO: 2.

4. The compound according to claim 1, wherein the compound is a biomarker of multiple sclerosis.

5-7. (canceled)

8. An in vitro method for predicting the risk of contracting or developing multiple sclerosis in a subject, comprising detecting a compound selected from the group consisting of:
a) a nucleic acid coding for a variant of the Epstein Barr nuclear antigen 2 (EBNA2), comprising:
the sequence coding for a nuclear antigen 2 of the sub-type 1.2 or variants thereof, having at least one substitution of the triplet coding for one or more amino acids selected from the group consisting of: aa. 134, aa. 236, aa. 245 and aa. 267 of SEQ ID NO: 2,
b) a messenger RNA transcribed from said nucleic acid, and
c) a protein coded by said nucleic acid in a biological sample isolated from the subject.

9. The method according to claim 8, wherein the presence of said nucleic acid is detected through DNA genotyping.

10. The method according to claim 8, wherein the detection of the presence of the sequence coding for the nuclear antigen 2 of the sub-type 1.3 B or of the protein coded by it is an indication of low risk.

11. An in vitro method for screening and/or for the diagnosis and/or prognosis of multiple sclerosis in a subject comprising detecting presence of a compound selected from the group consisting of:
a) a nucleic acid coding for a variant of the Epstein Barr nuclear antigen 2 (EBNA2), comprising:
the sequence coding for a nuclear antigen 2 of the sub-type 1.2 or variants thereof, having at least one substitution of the triplet coding for one or more amino acids selected from the group consisting of: aa. 134, aa. 236, aa. 245 and aa. 267 of SEQ ID NO: 2,
b) a messenger RNA transcribed from said nucleic acid, and
c) a protein coded by said nucleic acid, in a biological sample isolated from the subject.

12. The method according to claim 11, wherein the detection of the presence of the nucleic acid is carried out through DNA genotyping.

13. The method according to claim 11, wherein the subject in which the presence of the compound has been detected is then subjected to further methods for diagnosis and/or prognosis of multiple sclerosis.

14. The method according to claim 8, wherein the biological sample is selected from the group consisting of blood cells, biological fluids, serum, plasma, saliva or cerebrospinal fluid, cerebral tissue, and epithelial cells.

15. A kit for predicting the risk of contracting or developing multiple sclerosis and/or for screening and/or diagnosis and/or prognosis of multiple sclerosis comprising detection means for said compound of claim 8 and optionally control means.

16. The kit according to claim 15, wherein the detected compound is a nucleic acid and the kit further comprises means for amplifying said nucleic acid.

17. (canceled)
